(19) United States
(12) Patent Application Publication
(10) Pub. No.: US 2023/0404783 A1
(43) Pub. Date: Dec. 21, 2023

(54) IN-VIVO INDWELLING STENT AND STENT DELIVERY SYSTEM

(71) Applicant: Terumo Kabushiki Kaisha, Tokyo (JP)

(72) Inventors: Hiroshi YAMAGUCHI, Ashigarakami-gun (JP); Taishi NIIMI, Ashigarakami-gun (JP); Takashi KITAOKA, Ashigarakami-gun (JP); Yukiko TANABE, Ashigarakami-gun (JP)

(73) Assignee: TERUMO KABUSHIKI KAISHA, Tokyo (JP)

(21) Appl. No.: 18/460,795

(22) Filed: Sep. 5, 2023

Related U.S. Application Data
(63) Continuation of application No. PCT/JP2021/048280, filed on Dec. 24, 2021.

(30) Foreign Application Priority Data
Mar. 30, 2021 (JP) 2021-056740

(51) Int. Cl. A61F 2/915; A61F 2/966

(52) U.S. Cl. CPC A61F 2/915; A61F 2/966

(57) ABSTRACT

In an in-vivo indwelling stent, a plurality of annular bodies formed in a circular shape with linear components are arranged in an axial direction, and adjacent annular bodies are connected by connection portions. Each of the annular bodies includes connection linear portions that connect one end side bent portions and other end side bent portions. One of the connection linear portions of the annular bodies located in a central portion of the stent includes axial linear portions that have one end side vertices of the one end side bent portions as starting ends and extend so as to be parallel to the axial direction of the stent, and bent portions that have starting ends at terminal ends of the axial linear portions and extend in a circumferential direction of the stent.

### CROSS-REFERENCES TO RELATED APPLICATIONS

This application is a continuation of International Application No. PCT/JP2021/048280 filed on Dec. 24, 2021, which claims priority to Japanese Application No. 2021-056740 filed on Mar. 30, 2021, the entire content of both of which is incorporated herein by reference.

### TECHNOLOGICAL FIELD

The present disclosure generally relates to an in-vivo indwelling stent and a stent delivery system to be used for improving stenosis or occlusion generated in a body lumen such as a blood vessel, a bile duct, a trachea, an esophagus, or a urethra.

### BACKGROUND DISCUSSION

An in-vivo indwelling stent is used for treating various diseases caused by stenosis or occlusion of a blood vessel or other lumens in a living body. The stent is formed in a tubular shape to expand the stenotic or occluded site and secure an expanded inner diameter at the stenotic or occluded site.

Hereinafter, a blood vessel will be described as an example, but the present disclosure is not limited to a blood vessel.

The stent has a small diameter at the time of insertion so as to be inserted into the body from outside the body, expands at a target stenosis or occlusion site to increase the diameter, and holds a lumen in the expanded inner diameter as it is.

As the stent, a metal wire material or a cylindrical stent obtained by processing a metal tube is generally used. The stent is attached to a catheter, or the like, in a thinned state, inserted into a living body, expanded by some method at a target site, and fixed in close contact with an inner wall of a lumen to maintain a shape of the lumen.

Stents are distinguished into self-expanding stents and balloon-expanding stents by functions and indwelling methods. A balloon-expanding stent itself has no expansion function. After the stent mounted on the balloon is inserted into a target site, the balloon is expanded, and the stent is expanded (plastically deformed) by expansion force of the balloon and fixed in close contact with an inner surface of a target lumen. The balloon-expanding type of stent requires work of expanding the stent as described above. On the other hand, a self-expanding stent has an expansion function in the stent itself and is inserted into a living body in a thin and shrunk state, opened at a target site to return to its original expanded state and fixed in close contact with an inner wall of a lumen to maintain a shape of the lumen.

Currently, a stent is indwelled to return a blood vessel that has been stenosed for some reason to its original patent state (i.e., open and unobstructed state). A self-expanding stent is mostly used in a peripheral region such as a blood vessel of the lower limb and carotid. In a coronal region and a cerebrovascular region, a stent capable of reducing a diameter at the time of insertion is required.

The applicant of the present application proposes Japanese Patent Application Publication No. 2011-115634 A. A stent 1 of Japanese Patent Application Publication No. 2011-115634 A includes connection portions 5 in which a plurality of annular units 4 each including a plurality of annular linear bodies 2 and a connection portion 3 are arranged in an axial direction of the stent. Starting ends and terminal ends of the connection portions 5 are connected to between vertices of bent portions of a circular filament.

In Japanese Patent Application Publication No. 2011-115634 A, as described above, the starting ends and the terminal ends of the connection portions 5 are connected to between the vertices of the bent portions of the circular filament. In the connection portion, the circular filament extending in a circumferential direction is bent, but is connected to the connection portion before and after the bent portion and intersects in a cross shape at the connection portion as an intersection. In this stent, it was difficult to reduce a diameter at the connection portion at the time of diameter reduction.

### SUMMARY

An in-vivo indwelling stent is disclosed, which is a tubular in-vivo indwelling stent in which annular bodies formed with wavy and annular linear components including a plurality of one end side bent portions having vertices on one end side in an axial direction and a plurality of other end side bent portions having vertices on the other end side in the axial direction of the stent are arranged in a plurality of axial directions, and adjacent annular bodies are connected by connection portions, and which has favorable radial compression property and sufficient expansion force, and a stent delivery system.

An in-vivo indwelling stent in which a plurality of annular bodies each formed in an annular shape (or a circular shape) with one endless linear component are arranged in an axial direction, and adjacent annular bodies are connected by connection portions, each of the annular bodies including a plurality of one end side bent portions located on one end side in the axial direction of the stent, a plurality of other end side bent portions located on the other end side in the axial direction of the stent, and a plurality of connection linear portions that connect the one end side bent portions and the other end side bent portions, further, in an expanded state of the stent, one of the connection linear portions of the plurality of annular bodies located in a central portion of the stent including axial linear portions having one end side vertices of the one end side bent portions as starting ends, extending so as to be parallel to the axial direction of the stent as compared with the other portion of the connection linear portions, and having a shorter axial length than an axial length of the annular bodies; and bent portions having starting ends at terminal ends of the axial linear portions, extending in a circumferential direction of the stent and in a direction away from the one end side vertices, and having terminal ends connected to a remaining portion of the one of the connection linear portions, and the connection portions connecting the terminal ends of the axial linear portions and the one end side vertices of the annular body adjacent to the annular body having the axial linear portions on the other end side in the axial direction of the stent.

A stent delivery system including: a sheath; the stent stored in a distal end portion of the sheath; and a shaft configured to be inserted into the sheath and to be released the stent from a distal end of the sheath, in which the stent is formed in a substantially cylindrical shape, configured to be compressed in a central axis direction at the time of in-vivo insertion, and to expand outward to restore a shape before compression at the time of in-vivo indwelling.

An in-vivo indwelling stent comprising: a plurality of annular bodies formed in a circular shape arranged in an axial direction, the plurality of annular bodies including adjacent annular bodies being connected by connection portions; the plurality of annular bodies including a plurality of one end side bent portions located on one end side in the axial direction of the stent, a plurality of other end side bent portions located on the other end side in the axial direction of the stent, and a plurality of connection linear portions that connect the one end side bent portions and the other end side bent portions; one of the plurality of connection linear portions of the plurality of annular bodies located in a central portion of the stent includes axial linear portions having one end side vertices of the one end side bent portions as starting ends, extending so as to be parallel to the axial direction of the stent as compared with the other portion of the connection linear portions, and having a shorter axial length than an axial length of the plurality of annular bodies, and bent portions having starting ends at terminal ends of the axial linear portions, extending in a circumferential direction of the stent and in a direction away from the one end side vertices, and having terminal ends connected to a remaining portion of the one of the connection linear portions; the connection portions connecting the terminal ends of the axial linear portions and the one end side vertices of the annular body adjacent to the annular body having the axial linear portions on the other end side in the axial direction of the stent; and wherein the connection portions include: connection portion parallel portions having one ends at the terminal ends of the axial linear portions; and connection portion inclined portions extending obliquely in the circumferential direction and the axial direction of the stent from the other ends of the connection portion parallel portions, and terminal ends of the connection portion inclined portions are connected to one end side vertices other than the closest one end side vertices of the adjacent annular body.

An in-vivo indwelling stent comprising: a plurality of annular bodies including adjacent annular bodies being connected by connection portions; the plurality of annular bodies including a plurality of one end side bent portions located on one end side in the axial direction of the stent, a plurality of other end side bent portions located on the other end side in the axial direction of the stent, and a plurality of connection linear portions that connect the one end side bent portions and the other end side bent portions; in an expanded state of the stent, one of the plurality of connection linear portions of the plurality of annular bodies located in a central portion of the stent includes axial linear portions having one end side vertices of the one end side bent portions as starting ends, extending so as to be parallel to the axial direction of the stent as compared with the other portion of the connection linear portions, and having a shorter axial length than an axial length of the plurality of annular bodies, and bent portions having starting ends at terminal ends of the axial linear portions, extending in a circumferential direction of the stent and in a direction away from the one end side vertices, and having terminal ends connected to a remaining portion of the one of the connection linear portions; the connection portions connecting the terminal ends of the axial linear portions and the one end side vertices of the annular body adjacent to the annular body having the axial linear portions on the other end side in the axial direction of the stent, the connection portions including: connection portion parallel portions having one ends at the terminal ends of the axial linear portions and extending substantially parallel to the axial direction of the stent; and connection portion inclined portions extending obliquely in the circumferential direction and the axial direction of the stent from the other ends of the connection portion parallel portions, and terminal ends of the connection portion inclined portions are connected to one end side vertices other than the closest one end side vertices of the adjacent annular body; and wherein the axial linear portions and the connection portion parallel portions of the connection portions have a continuous linear shape.

### BRIEF DESCRIPTION OF THE DRAWINGS

FIG. 1 is a developed view at the time of manufacturing an in-vivo indwelling stent according to an embodiment of the present disclosure.

FIG. 2 is a partially enlarged view of the in-vivo indwelling stent of FIG. 1.

FIG. 3 is an explanatory view for explaining an annular body of the in-vivo indwelling stent of FIG. 1.

FIG. 4 is an external view of the in-vivo indwelling stent of FIG. 1 at the time of expansion.

FIG. 5 is an external view of the in-vivo indwelling stent of FIG. 1 at the time of diameter reduction.

FIG. 6 is a partially enlarged view of the in-vivo indwelling stent of FIG. 5.

FIG. 7 is an external view of an in-vivo indwelling stent of another embodiment of the present disclosure at the time of expansion.

FIG. 8 is a front view of a stent delivery system of the embodiment of the present disclosure.

FIG. 9 is a partially broken enlarged view of a distal end portion of the stent delivery system illustrated in FIG. 8.

### DETAILED DESCRIPTION

Set forth below with reference to the accompanying drawings is a detailed description of embodiments of an in-vivo indwelling stent.

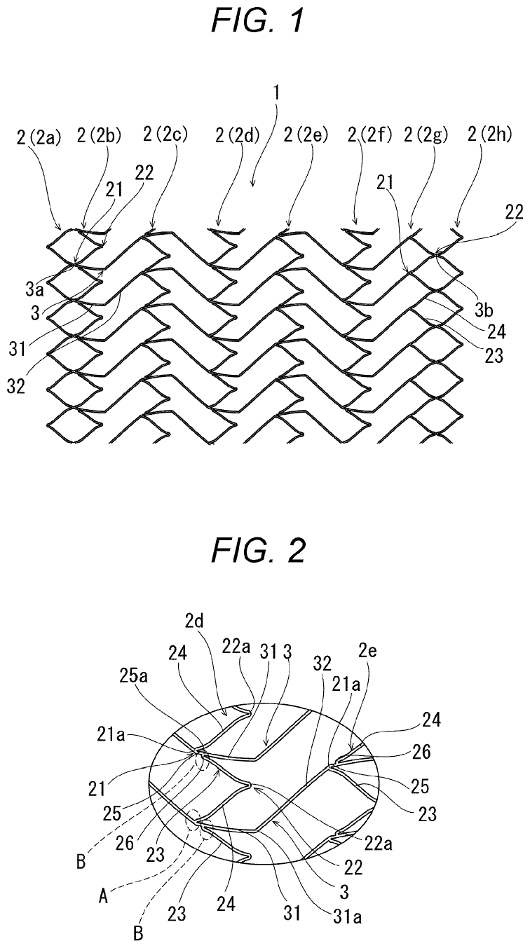

As illustrated in FIGS. 1 and 2, an in-vivo indwelling stent 1 of the present disclosure is a stent for in-vivo indwelling in which a plurality of annular bodies 2 each formed in an annular shape (or a circular shape) with one endless linear component (strut) are arranged in the axial direction, and adjacent annular bodies 2 (2a, 2b, 2c, 2d, 2e, 2f, 2g, 2h) are connected by connection portions 3.

The annular bodies 2a, 2b, 2c, 2d, 2e, 2f, 2g, and 2h include a plurality of one end side bent portions 21 located on one end side (left side in FIG. 1) in an axial direction of the stent, a plurality of the other end side bent portions 22 located on the other end side (right side in FIG. 1, a direction of insertion of the stent into a living body) in the axial direction of the stent, and a plurality of connection linear portions 23, 24 that connect the one end side bent portions 21 and the other end side bent portions 22.

Furthermore, in an expanded state of the stent 1, one (for example, 23) of the connection linear portions (including an axial linear portion 25 and a bent portion 26 to be described later) 23, 24 of the plurality of annular bodies 2 located in a central portion (i.e., between the one end side and the other end side in the axial direction) of the stent includes: axial linear portions 25 having one end side vertices 21a of the one end side bent portions 21 as starting ends, extending so as to be parallel to the axial direction of the stent 1 as compared with the other portion of the connection linear portions 23, 24, and having a shorter axial length than an axial length of the annular bodies 2; and bent portions 26 having starting ends at terminal ends 25a of the axial linear portions 25, extending in a circumferential direction of the stent 1 and in a direction away from the one end side vertices 21a, and having the terminal ends connected to the remaining portion of the one of the connection linear portions 23 (24). The connection portions 3 connect the terminal ends 25a of the axial linear portions 25 and the one end side vertices 21a of the annular body 2 adjacent to the annular body 2 having the axial linear portions 25 on the other end side in the axial direction of the stent 1.

The stent 1 can be effectively used as a stent for expansion of a cerebral artery.

The stent 1 of this embodiment is a so-called self-expanding stent that is formed in a substantially cylindrical shape, is compressed in a central axis direction at the time of in-vivo insertion and expands outward to restore a shape before compression at the time of in-vivo indwelling.

As illustrated in FIGS. 1 to 6, the stent 1 of this embodiment has a form in which a plurality of annular bodies 2a, 2b, 2c, 2d, 2e, 2f, 2g, and 2h are arranged in parallel in the axial direction so as to be adjacent to each other in the axial direction, and adjacent annular bodies are connected. Each of the annular bodies 2b, 2c, 2d, 2e, 2f, 2g, and 2h is formed with a wavy and circular linear component including a plurality of one end side bent portions 21 having the one end side vertices 21a in the axial direction and a plurality of other end side bent portions 22 having the other end side vertices 22a in the axial direction of the stent 1.

The number of the annular bodies 2 in the stent 1 can be, for example, preferably 5 to 30, and more preferably 6 to 20. The number of annular bodies 2 in the stent 1 of this embodiment, for example, is eight.

As illustrated in FIG. 1, the annular bodies 2a, 2b, 2c, 2d, 2e, 2f, 2g, and 2h in the stent 1 of this embodiment include a plurality of one end side bent portions 21 and other end side bent portions 22 having substantially the same pitch. The number of the one end side bent portions 21 (or the other end side bent portions 22) in each of the annular bodies 2a, 2b, 2c, 2d, 2e, 2f, 2g, and 2h can be, for example, preferably 4 to 10, and more preferably 4 to 8.

Then, in the stent 1 of this embodiment, as illustrated in FIGS. 1, 2, and 4, at the time of formation and in the expanded state of the stent 1, one of the connection linear portions 23, 24 of the plurality of annular bodies 2 located in the central portion of the stent includes: the axial linear portions 25 having the one end side vertices 21a of the one end side bent portions 21 as starting ends, extending parallel to the axial direction of the stent 1 as compared with the other portion of the connection linear portions 23, 24, and having a shorter axial length than an axial length of the annular bodies 2; and the bent portions 26 having the starting ends at the terminal ends 25a of the axial linear portions 25, extending in the circumferential direction of the stent 1 and in a direction away from the one end side vertices 21a, and having the terminal ends connected to the remaining portion of the one of the connection linear portions 23, 24.

The axial linear portions 25 extend in the axial direction and the other end direction of the stent 1 from the inside of the one end side vertices 21a of the annular body 2 (a portion located on the most one end side in the one end side bent portions). In addition, the bent portion 26 includes a circumferential extension portion that is bent from the terminal end 25a of the axial linear portion 25 and extends short in the circumferential direction of the stent, and a bent portion that is formed at a terminal end portion of the circumferential extension portion and obliquely extends in the other end direction of the stent 1. The terminal end of the bent portion of the bent portion 26 is connected to the remaining portion of the connection linear portion 23.

The number of the annular bodies 2 provided with the axial linear portions 25 and the bent portions 26 as described above and located at the central portion of the stent can be, for example, preferably 5 to 30, and more preferably 6 to 20. The number of the annular bodies 2 of the above type in the stent 1 of this embodiment, for example, is five. In addition, all the annular bodies 2 located in the central portion of the stent are preferably annular bodies including the axial linear portions 25 and the bent portions 26.

Furthermore, in the stent 1 of this embodiment, as illustrated in FIG. 1, in the annular bodies 2b, 2c, 2d, 2e, and 2f including the axial linear portions 25 and the bent portions 26 as described above, all of the connection linear portions 23, 24 include the axial linear portions 25 and the bent portions 26, and the connection portions 3 are connected to the terminal ends of all the axial linear portions 25.

In the stent 1 of this embodiment, the axial linear portions 25 are straight-line portions elongated along the axial direction of the stent 1 during the stent is compressed in a stent delivery system. In addition, the axial linear portion 25 extends so as to be parallel to the axial direction of the stent 1 as compared with other portions of the connection linear portion 23 (24). Specifically, the axial linear portion 25 is substantially parallel to the axial direction of the stent 1, but slightly inclined with respect to the central axis of the stent.

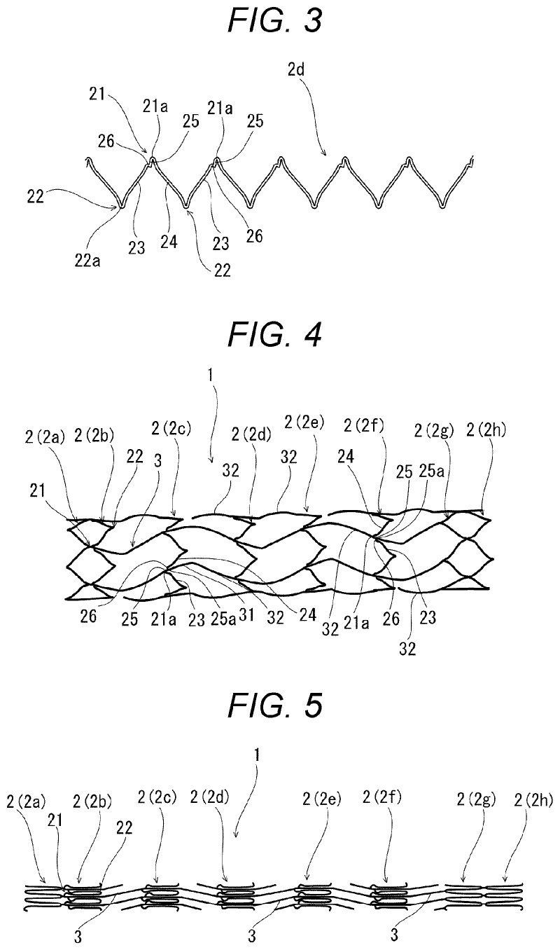

As illustrated in FIG. 3, the one end side bent portions 21 of the annular bodies 2 (2b, 2c, 2d, 2e, 2f) including the axial linear portions 25 and the bent portions 26 include first inflection points formed by the vertices 21a and second inflection points formed by the bent portions 26. The second inflection points (bent portions 26) are located closer to the other end side than the first inflection points (vertices 21a) having a predetermined length (specifically, a length of the axial linear portions 25 is equal to a length of the axial linear portions). A plurality of one end side bent portions 21 including the axial linear portions 25 and the bent portions 26 are arranged in the circumferential direction. As illustrated in FIG. 3, the other end side bent portions 22 include single inflection points formed by the vertices 22a, and a plurality of the other end side bent portions 21 are arranged in the circumferential direction. Thus, in the annular body 2 including the axial linear portion 25 and the bent portion 26, shapes of the one end side bent portion 21 and the other end side bent portion 22 are different.

Further, as illustrated in FIGS. 1 to 3, the connection linear portions 23, 24 (the connection linear portion 23 in the annular bodies 2b, 2d, and 2f) including the axial linear portions 25 and the bent portions 26 include the axial linear portions 25 and the bent portions 26, and thus, have a longer length of the linear components (struts) than that of the connection linear portions 23, 24 (the connection linear portion 24 in the annular bodies 2b, 2d, and 2f) which do not include the axial linear portions 25 and the bent portions 26 and which connect the one end side vertices 21a of the one end side bent portions 21 and the other end side vertices 22a of the other end side bent portions 22.

Further, as illustrated in FIGS. 1 to 3, the connection linear portions 23, 24 (the connection linear portion 23 in the annular body 2d) of the portion that connects the terminal ends 25a of the axial linear portions 25 and the other end side vertices 22a of the other end side bent portions 22 (including the bent portions 26) has a shorter length of the linear components (struts) than that of the connection linear portions 23, 24 (the connection linear portion 24 in the annular body 2d) that directly connect the one end side vertices 21a of the one end side bent portions 21 and the other end side vertices 22a of the other end side bent portions 22. As a result, even when the stent is indwelled in the bent portion, it is possible to help reduce a risk that the other end side vertices 22a and connection portion inclined portions 32 come into contact with each other and the stent is deformed.

A distance between the one end side vertex 21a and the terminal end 25a of the axial linear portion 25 or the starting end of the bent portion 26 (the length of the axial linear portion 25) is preferably, for example, 1/16 to ½, of the length of the connection linear portion (the connection linear portion not including the axial linear portion 25 and the bent portion 26) extending from the one end side vertex 21a, and more preferably ⅛ to ¼, of the length of the connection linear portion extending from the one end side vertex 21a. The axial length of the axial linear portion 25 can be, for example, preferably 1/16 to ½ of the axial length of the annular body 2, and more preferably ⅛ to ¼ of the axial length of the annular body 2.

Further, in the stent 1 of this embodiment, the annular body 2 located at the central portion of the stent 1 described above includes a plurality of bent portions 26 as illustrated in FIGS. 1 to 3 and the bent portions 26 extend in the same circumferential direction.

Furthermore, in the stent 1 of this embodiment, as illustrated in FIGS. 1 and 2, the one end side bent portions 21 of the annular body 2 (2d) including the axial linear portions 25 and the bent portions 26 include first branch portions A formed by the other end portions of the connection portions 3, the vertices 21a, and the axial linear portions 25, and second branch portions B formed by the axial linear portions 25, one end portions of the connection portions 3, and the bent portions 26 at positions shifted to the other end side by a predetermined length (by a length of the axial linear portion 25) from the first branch portions A. The connection linear portions 23, 24 (the connection linear portion 24 in the annular body 2d) having the first branch portions A and the connection linear portions 23, 24 (the connection linear portion 23 in the annular body 2d) having the second branch portions B are connected at the other end side bent portions 22.

In other words, in the stent 1 of the present disclosure, the connection linear portions 23, 24 (the connection linear portion 24 in the annular body 2d) including the one end side bent portions 21 and the connection linear portions 23, 24 (the connection linear portion 23 in the annular body 2d) including the axial linear portions 25 and the bent portions 26 are connected at the other end side bent portions 22. There is no connection portion between the two connection linear portions 23, 24 adjacent the other end side bent portions 22.

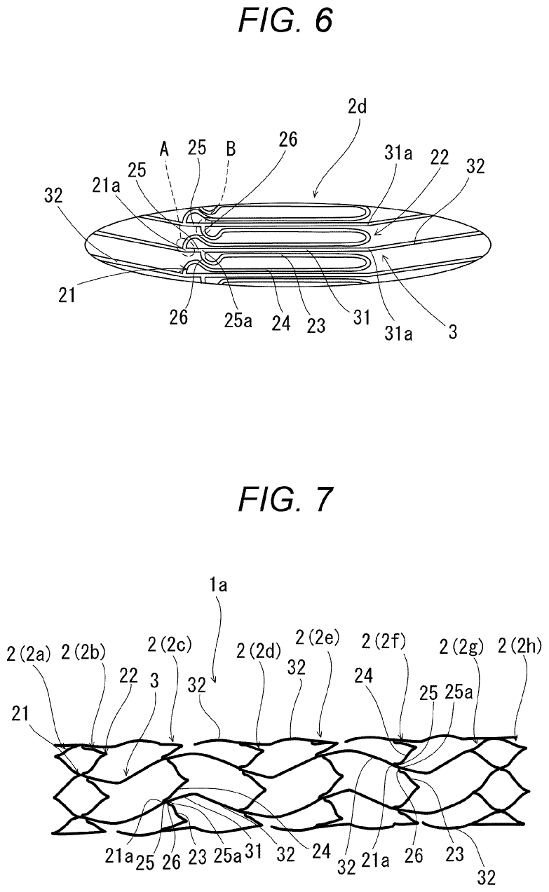

The first branch portion A and the second branch portion B are branch portions and do not intersect in a cross shape. As a result of the first branch portions A and the second branch portions B being shifted to the other end side by a predetermined length (by the length of the axial linear portion 25), as illustrated in FIGS. 5 and 6, radial deformability (diameter reduction) at the one end side bent portions of the annular bodies having the first branch portions A and the second branch portions B can be improved at the time of compression (diameter reduction) of the stent.

Specifically, when the stent is compressed (reduced in diameter), the stent 1 is in a state as schematically illustrated in FIGS. 5 and 6. When the stent 1 is compressed in the radial direction, as illustrated in FIG. 6, the linear components (struts) of the connection linear portions 23, 24 (the connection linear portions 24 in the annular body 2d) having the second branch portions B are brought close to the linear components (struts) of the connection linear portions 23, 24 (the connection linear portions 23 in the annular body 2d) having the first branch portions A that are directly connected to the connection linear portions 23, 24.

The deformed first branch portions A form curved portions extending in the first circumferential direction, and similarly, the deformed second branch portions B form curved portions extending in a direction opposite to the first circumferential direction. The second branch portions B are shifted from the first branch portions A to the other end side by a predetermined length (by the length of the axial linear portion 25), and thus, the curved portions formed by the deformed second branch portions B do not overlap the curved portions formed by the deformed first branch portions A and are located on the other end side. In particular, in the stent 1 of this embodiment, the curved portions formed by the deformed second branch portions B enter the other end side of the curved portions formed by the deformed first branch portions A.

For this reason, the first branch portions A and the second branch portions B in the stent 1 are less likely to become obstacles at the time of diameter reduction of the stent 1, so that favorable radial deformability (diameter reduction) can be obtained at the time of compression (diameter reduction) of the stent.

Furthermore, in the stent 1 of this embodiment, as illustrated in FIGS. 1 and 2, in the adjacent annular bodies 2 located in the central portion of the stent 1 described above, the bent portions 26 adjacent in the axial direction of the stent 1 extend in different circumferential directions. In the stent of this embodiment, the connection linear portions (the connection linear portions 24 in the annular bodies 2b, 2d, and 2f) including the one end side bent portions 21 and the connection linear portions (the connection linear portions 23 in the annular bodies 2b, 2d, and 2f) including the axial linear portions 25 and the bent portions 26 are connected at the other end side bent portions 22. The connection linear portions (the connection linear portions 23 in the annular bodies 2c and 2e) including the one end side bent portions 21 and the connection linear portions (the connection linear portions 24 in the annular bodies 2c and 2e) including the axial linear portions 25 and the bent portions 26 are connected at the other end side bent portions 22.

As a result of this, uniform expansion force in the entire stent can be exerted. Note that, although the above is preferable, as in the stent 1a illustrated in FIG. 7, in the adjacent annular bodies 2 located in the central portion of the stent 1a, all the bent portions 26 adjacent in the axial direction of the stent 1a may extend in the same circumferential direction.

As illustrated in FIGS. 1 to 6, the stent 1 includes the connection portions 3 that connect the terminal ends 25a of the axial linear portions 25 and the one end side vertices 21a of the annular body 2 adjacent to the annular body 2 having the axial linear portions 25 on the other end side in the axial direction of the stent 1. In the stent 1 of this embodiment, the terminal ends 25a of all the axial linear portions 25 of the annular bodies 2 having the axial linear portions 25 are connected to the connection portions 3.

In the stent 1 of this embodiment, the connection portions 3 includes connection portion parallel portions 31 having one ends at the terminal ends 25a of the axial linear portions 25 and extending substantially parallel to the axial direction of the stent 1, and connection portion inclined portions 32 extending obliquely in the circumferential direction and the axial direction of the stent 1 from the other ends of the connection portion parallel portions 31, and terminal ends of the connection portion inclined portions 32 are connected to one end side vertices 21a other than the closest one end side vertex of the adjacent annular body 2.

Specifically, in the stent 1 of this embodiment, the axial linear portions 25 and the connection portion parallel portions 31 of the connection portions 3 have a continuous linear shape. In addition, in the stent 1 of this embodiment, the terminal ends of the connection portion inclined portions 32 are connected to the one end side vertices 21a adjacent in a circumferential direction to the closest one end side vertices of the adjacent annular body 2.

In the stent 1 of this embodiment, as illustrated in FIG. 1, all the connection portion inclined portions 32 between the adjacent annular bodies 2 are inclined in the same circumferential direction. In particular, in the stent 1, all the connection portion inclined portions 32 between the adjacent annular bodies 2 are substantially parallel. Further, the connection portion inclined portions 32 adjacent to each other in the axial direction of the stent are inclined in different directions in the circumferential direction of the stent 1. In the stent 1, the connection portion inclined portions 32 adjacent to each other in the axial direction are inclined in different circumferential directions and are inclined at substantially the same angle with respect to the central axis of the stent 1.

In the stent 1 of this embodiment, the connection portion inclined portions 32 of the connection portions 3 are inclined in the axial direction of the stent and curved in the circumferential direction. Thus, as illustrated in FIG. 4 which is an external view when the stent 1 is expanded, the connection portion inclined portions 32 are in a curved state in which the central portions of the connection portion inclined portions 32 dilate outward when the stent 1 is expanded. The stent 1 includes convex portions that are positioned between adjacent annular bodies and protrude to the outside of the stent in an expanded state of the stent 1. The convex portions are formed by intermediate portions of the connection portions protruding outward. In addition, a protrusion amount increases from both ends of the connection portions toward the central portions, and the central portions are vertices of the convex portions. In a case where a space between the convex portions is regarded as a recess, irregularities are generated outside the stent, and a period of the irregularities in the axial direction is a distance in the axial direction between the one end side vertices or between the other end side vertices of the adjacent annular bodies. Thus, adhesion of the stent 1 with the inserted living body site can be improved, and movement of the stent 1 can be restricted with the inserted living body site. The axial linear portion 25 and the connection portion 3 constitute one wavy linear portion extending in the axial direction. In order to ensure sufficient adhesion of the stent with the living body site, a height of the convex portions (distance between the irregularities) is desirably, for example, equal to or greater than 50 μm.

In the stent 1 of this embodiment, terminal ends 31a of the connection portion parallel portions 31 of the connection portions 3 are located on the same side as the other end side vertices 22a of the annular bodies 2 including the axial linear portions 25 to which one ends of the connection portions 3 are connected or slightly closer to the other end side of the stent 1 than the other end side vertices 22a in the axial direction of the stent 1 in the expanded state of the stent 1.

In this way, when the diameter of the stent is reduced, the connection portion inclined portions 32 connected to the terminal ends 31a of the connection portion parallel portions 31 of the connection portions 3 do not abut on the other end side bent portions 22 of the annular bodies 2, so that the diameter is favorably reduced. The terminal ends 31a of the connection portion parallel portions 31 of the connection portions 3 are preferably located closer to the other end side of the stent 1 than the other end side vertices 22a of the annular bodies 2.

In addition, it is preferable that the terminal ends 31a of the connection portion parallel portions 31 of the connection portions 3 are located on the same side as the other end side vertices 22a of the annular bodies 2 including the axial linear portions 25 to which one ends of the connection portions 3 are connected or slightly closer to one end side of the stent 1 than the other end side vertices 22a in the axial direction of the stent 1 in a contracted state of the stent 1.

In this way, when the stent is contracted and stored in the sheath, the connection portion inclined portions 32 become close to parallel in the axial direction, the axial force is rather easily transmitted, so that rigidity in the axial direction can be enhanced.

As illustrated in FIG. 1, the stent 1 of this embodiment includes: one end portion annular body connection portions 3a that connect the other end side vertices 22a of the annular body 2a located at one end and the one end side vertices 21a of the annular body 2b adjacent to the annular body 2a located at one end; and other end portion annular body connection portions 3b that connect the one end side vertices 21a of the annular body 2h located at the other end and the other end side vertices 22a of the annular body 2g adjacent to the annular body 2h located at the other end. Thus, both ends of the stent 1 are in a so-called closed cell state and have relatively high axial rigidity.

In addition, in the stent 1 of this embodiment, in the annular bodies 2b, 2c, 2d, 2e, 2f, 2g, and 2h excluding the annular body 2a located at one end of the stent 1, the vertices 21a of all the one end side bent portions 21 of the adjacent annular bodies 2b, 2c, 2d, 2e, 2f, 2g, and 2h are connected to the connection portions 3, 3a, and 3b. Thus, the stent 1 of this embodiment does not have the one end side bent portion 21 in which the vertex 21a is a free end except for the annular body 2a located at one end of the stent 1. Furthermore, in the stent 1 of this embodiment, each of the annular bodies 2b, 2c, 2d, 2e, 2f, and 2h excluding the annular bodies 2a and 2g includes a plurality of other end side bent portions 22 in which the other end side vertices 22a are free ends.

Further, in the stent of this embodiment, a plurality of single wavy linear portions formed by the axial linear portions 25 and the connection portions 3 are provided substantially uniformly in the circumferential direction. As a result, the entire stent is deformed well at the time of compression and tension of the stent, and favorable blood vessel followability can be obtained in a case where the stent is indwelled in a bent blood vessel.

The axial length of the connection portion 3 at the time of expansion of the stent is preferably, for example, 5/4 to 3 times the axial length of the annular body 2, and more preferably 3/2 to 2 times the axial length of the annular body 2. The axial length of the connection portion parallel portion 31 of the connection portion 3 at the time of expansion of the stent is preferably, for example, ½ to 2 times the axial length of the annular body 2, and more preferably ¾ to 5/4 times the axial length of the annular body 2. The axial length of the connection portion inclined portion 32 of the connection portion 3 is preferably, for example, ¼ to 2 times the axial length of the annular body 2, and more preferably ½ to 3/2 times the axial length of the annular body 2. In addition, the axial length of the connection portion inclined portion 32 of the connection portion 3 at the time of expansion of the stent is preferably longer than the axial length of the connection portion parallel portion 31. The inclination angle of the connection portion inclined portion 32 of the connection portion 3 with respect to the central axis of the stent 1 is preferably, for example, 35 degrees to 65 degrees, and more preferably 40 degrees to 60 degrees.

The stent of the present embodiment can be reduced in diameter and can be stably indwelled even in the bent portion, and thus, the stent is suitable for being indwelled in a cerebral artery having a small inner diameter and many bent portions and is suitable as a stent for treating cerebral artery stenosis.

A superelastic metal is suitable as a constituent material of the stent. As the superelastic metal, a superelastic alloy is suitably used. The superelastic alloy in the disclosure is generally called a shape memory alloy and exhibits superelasticity at least at a biological temperature (around 37° C.). Particularly preferably, a superelastic alloy such as a Ti—Ni alloy including Ni of 49 atomic % to 53 atomic %, a Cu—Zn alloy including Zn of 38.5 weight % to 41.5 weight %, a Cu—Zn—X alloy (X=Be, Si, Sn, Al, Ga) including X of 1 weight % to 10 weight %, or a Ni—Al alloy including Al of 36 atomic % to 38 atomic % is suitably used. The Ti—Ni alloy is particularly preferable. In addition, mechanical characteristics can be appropriately changed by using a Ti—Ni—X alloy (X=Co, Fe, Mn, Cr, V, Al, Nb, W, B, etc.) in which a part of the Ti—Ni alloy is substituted with 0.01 weight % X to 10.0 weight % X, using a Ti—Ni—Y alloy (Y=Cu, Pb, Zr) in which a part of the Ti—Ni alloy is substituted with 0.01 weight % Y to 30.0 weight % Y, and selecting a cold working ratio or/and conditions of a final heat treatment. In addition, by selecting the cold working ratio and/or the conditions of the final heat treatment using the Ti—Ni alloy, the Ti—Ni—X alloy, or the Ti—Ni—Y alloy, the mechanical characteristics can be appropriately changed. Buckling strength (yield stress at loading) of the superelastic alloy used is 5 kgf/mm2 to 200 kgf/mm2 (22° C.), more preferably 8 kgf/mm2 to 150 kgf/mm2, and a restoring stress (yield stress at unloading) is 3 kgf/mm2 to 180 kgf/mm2 (22° C.), more preferably 5 kgf/mm2 to 130 kgf/mm2. Superelasticity as used herein means that even if a normal metal is deformed (bent, pulled, compressed) to a region where the metal is plastically deformed at an operating temperature, the metal is restored to a substantially pre-compressed shape without requiring heating after the deformation is released.

Then, as the stent 1, for example, in a case where the stent is used for expansion of a cerebral blood vessel, the diameter at the time of expansion (at the time of non-compression) is preferably, for example, about 0.5 mm to 6.0 mm, and more preferably 0.9 mm to 5.0 mm. In addition, a length of the stent at the time of expansion (at the time of non-compression) is preferably, for example, about 5 mm to 50 mm.

A thickness of the stent is preferably, for example, about 0.05 mm to 0.15 mm, and more preferably 0.06 mm to 0.13 mm. A width of a linear component constituting the stent is preferably, for example, about 0.04 mm to 0.15 mm, and more preferably 0.05 mm to 0.13 mm.

In addition, in the stent of the present disclosure, the stent may contain a physiologically active substance so as to be releasable. As a method of containing a physiologically active substance so as to be releasable, for example, there is a method of coating a surface of the stent with a polymer (for example, a biodegradable polymer) containing a physiologically active substance.

The biodegradable polymer is not particularly limited as long as the biodegradable polymer is enzymatically or non-enzymatically decomposed in vivo and the decomposed product does not exhibit toxicity, and for example, polylactic acid, polyglycolic acid, a polylactic acid-polyglycolic acid copolymer, polycaprolactone, a polylactic acid-polycaprolactone copolymer, a polyorthoester, polyphosphazene, a polyphosphoric acid ester, polyhydroxybutyric acid, polymalic acid, poly-a-amino acid, collagen, gelatin, laminin, heparan sulfate, fibronectin, vitronectin, chondroitin sulfate, hyaluronic acid, polypeptide, chitin, chitosan, and the like, can be used.

In addition, as the physiologically active substance, a substance that promotes melting or metabolism of thrombus or thrombus complex, a substance that suppresses increase in thrombus or thrombus complex, a substance that suppresses intimal thickening, an anticancer drug, an immunosuppressive agent, an antibiotic, an antirheumatic agent, an antithrombotic agent, an HMG-CoA reductase inhibitor, an ACE inhibitor, a calcium antagonist, an antilipemia agent, an anti-inflammatory agent, an integrin inhibitor, an antiallergic agent, an antioxidant, a GP IIb IIIa antagonist, a retinoid, a flavonoid and a carotenoid, a lipid improver, a DNA synthesis inhibitor, a tyrosine kinase inhibitor, an antiplatelet agent, a vascular smooth muscle proliferation inhibitor, a biological material, interferon, and an epithelial cell generated by genetic engineering are used. Further, a mixture of two or more kinds of the above-mentioned substances, and the like, may be used.

As the substance that promotes the melting or metabolism of thrombus or thrombus complex or the substance that suppresses increase in thrombus or thrombus complex, streptokinase, plasminogen activator, urokinase, stafinokinase, lumbrokinase, nattokinase, or an analog of streptokinase, plasminogen activator, urokinase, stafinokinase, lumbrokinase, or nattokinase can be used. In addition, as the substance that suppresses increase in thrombus or a thrombus complex, an antiplatelet drug represented by acetylsalicylic acid, ticlopidine, dipyridamole, cilostazol, beraprost Na, rimaprost alfathecus, ethyl icosapentoenate, salvogrelate hydrochloride, trapidyl, clopidogrel, prasugrel, and analogs thereof, or an anticoagulant represented by a GP IIb/IIIa antagonist, heparin, or warfarin potassium can be used.

Next, a stent delivery system of an embodiment of the present disclosure will be described using an embodiment illustrated in the drawings.

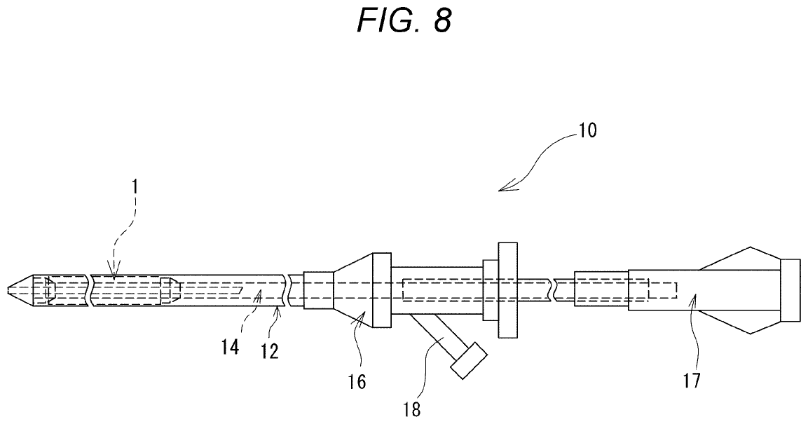

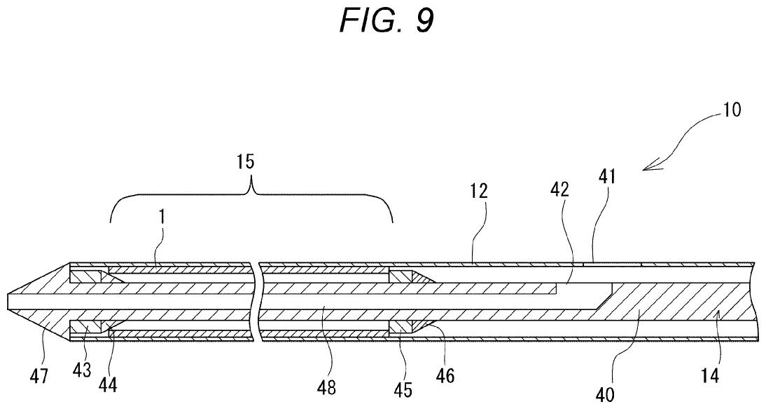

FIG. 8 is a partially omitted front view of the stent delivery system of the embodiment of the present disclosure. FIG. 9 is an enlarged longitudinal cross-sectional view of the vicinity of a distal end portion of the stent delivery system illustrated in FIG. 8.

A stent delivery system 10 of this embodiment includes the sheath 12, the stent 1 stored in a distal end portion of the sheath 12, and the inner tube 14 that is to be slidably inserted through the sheath 12 and releases the stent 1 from the distal end of the sheath 12.

In the stent delivery system 10 of this embodiment, as the stent 1, the above-described self-expanding stent is used which is formed in a cylindrical shape, is compressed in the central axis direction at the time of in-vivo insertion, and expands outward so as to be able to restore the shape before compression at the time of in-vivo indwelling.

As illustrated in FIG. 8, the stent delivery system 10 of this embodiment includes the sheath 12, the self-expanding stent 1, and the inner tube 14.

As illustrated in FIGS. 8 and 9, the sheath 12 is a tubular body and has openings at a distal end of the tubular body and a proximal end of the tubular body. The opening at the distal end functions as a release port of the stent 1 when the stent 1 is indwelled in a stenosed part in a body cavity. By sliding the sheath 12 toward the proximal end side, the stent 1 is released from the opening at the distal end, stress load is released, and the stent 1 expands and returns to the shape before compression. The distal end portion of the sheath 12 is a stent storage portion 15 that stores the stent 1 in the stent storage portion 15. In addition, the sheath 12 includes a side hole 41 provided on a proximal end side of the storage portion 15. The side hole 41 is provided for leading a guide wire to the outside.

An outer diameter of the sheath 12 is preferably, for example, about 0.4 mm to 4.0 mm, and more preferably 0.5 mm to 3.0 mm. An inner diameter of the sheath 12 is preferably, for example, about 0.3 mm to 2.0 mm. A length of the sheath 12 is preferably, for example, about 300 mm to 2500 mm, and more preferably, about 300 mm to 2000 mm.

As illustrated in FIG. 8, a sheath hub 16 is fixed to a proximal end portion of the sheath 12. The sheath hub 16 includes a sheath hub body and a valve body that is stored in the sheath hub body and slidably and liquid-tightly holds the inner tube 14. In addition, the sheath hub 16 includes a side port 18 branching obliquely rearward from the vicinity of the center of the sheath hub body. The sheath hub 16 preferably includes an inner tube lock mechanism that restricts movement of the inner tube 14.

As illustrated in FIGS. 8 and 9, the inner tube 14 includes a shaft-like inner tube main body 40, a distal end portion 47 provided at a distal end of the inner tube main body 40 and protruding from the distal end of the sheath 12, and an inner tube hub 17 fixed to a proximal end portion of the inner tube main body 40.

The distal end portion 47 is preferably formed in a tapered shape protruding from the distal end of the sheath 12 and gradually reducing in diameter toward the distal end as illustrated in FIG. 9. This configuration facilitates insertion into the stenosed part. In addition, it is preferable that the inner tube 14 is provided on the distal end side of the stent 1 and includes a stopper that inhibits movement of the sheath in a distal end direction. A proximal end of the distal end portion 47 can abut against the distal end of the sheath 12 and functions as the above-described stopper.

In addition, as illustrated in FIG. 9, the inner tube 14 includes two protrusion 43 and 45 for holding the self-expanding stent 1. The two protrusions 43 and 45 are preferably circular protrusions. A stent holding protrusion 43 is provided on the proximal end side of the distal end portion 47 of the inner tube 14. A stent extrusion protrusion 45 is provided on the proximal end side of the stent holding protrusion 43 by a predetermined distance. The stent 1 is disposed between the two protrusions 43 and 45. The two protrusions 43 and 45 have an outer diameter so that the protrusions 43 and 45 can abut against the compressed stent 1.

Thus, movement of the stent 1 to the distal end side is restricted by the protrusion 43, and movement to the proximal end side is restricted by the protrusion 45. By moving the sheath 12 to the proximal end side and moving the inner tube 14 to the distal end side, the stent 1 can be discharged from the sheath 12. Furthermore, as illustrated in FIG. 9, the proximal end side of the stent extrusion protrusion 45 preferably has a tapered portion 46 whose diameter gradually decreases toward the proximal end side. Similarly, as illustrated in FIG. 9, the proximal end side of the stent holding protrusion 43 preferably has a tapered portion 44 whose diameter gradually decreases toward the proximal end side.

In this manner, when the inner tube 14 protrudes from the distal end of the sheath 12 and the inner tube 14 is restored in the sheath 12 after the stent 1 is released from the sheath 12, the protrusions are prevented from being caught by the distal end of the sheath. As described above, the stent 1 of the present disclosure does not have the one end side bent portion 21 that is a free end except for the annular body 2a located at one end of the stent 1, and can be restored in the sheath 12 again even if the stent is exposed to some extent. The protrusions 43 and 45 may be formed with different members using X-ray contrast materials. As a result, a position of the stent can be accurately grasped under X-ray imaging, which facilitates procedure.

As illustrated in FIG. 9, the inner tube 14 includes a lumen 48 extending at least from the distal end to the proximal end side of the stent storage portion 15 of the sheath 12 and an inner tube side hole 42 communicating with the lumen 48 on the proximal end side of the stent storage portion. In the stent delivery system 10 of this embodiment, the lumen 48 terminates at a portion where the side hole 42 is formed. The lumen 48 is provided for inserting one end of the guide wire from the distal end of the stent delivery system 10, partially inserting the guide wire through the inner tube, and then leading the guide wire to the outside from a side surface of the inner tube. The side hole 42 of the inner tube is located slightly closer to the distal end side of the stent delivery system 10 from the side hole 41 of the sheath. The center of the side hole 42 of the inner tube is preferably 0.5 to 10 mm distal from the center of the side hole 41 of the sheath.

Note that the stent delivery system is not limited to the above-described type, and the lumen 48 may extend to the proximal end of the inner tube. In this case, the side hole 41 of the sheath is unnecessary.

The inner tube 14 penetrates inside of the sheath 12 and protrudes from the opening at the proximal end of the sheath 12. Note that as illustrated in FIG. 8, the inner tube hub 17 is fixed to a proximal end portion of the inner tube 14.

The stent of the present disclosure is an in-vivo indwelling stent in which a plurality of annular bodies each formed in a circular shape with one endless linear component are arranged in the axial direction, and adjacent annular bodies are connected by connection portions. The annular body includes a plurality of one end side bent portions located on one end side in the axial direction of the stent, a plurality of other end side bent portions located on the other end side in the axial direction of the stent, and a plurality of connection linear portions that connect the one end side bent portions and the other end side bent portions. Furthermore, in the expanded state of the stent, one of the connection linear portions of the plurality of annular bodies located in the central portion of the stent includes: axial linear portions having one end side vertices of the one end side bent portions as starting ends, extending so as to be parallel to the axial direction of the stent as compared with the other portion of the connection linear portions, and having a shorter axial length than an axial length of the annular bodies; and bent portions having starting ends at terminal ends of the axial linear portions, extending in a circumferential direction of the stent and in a direction away from the one end side vertices, and having terminal ends connected to the remaining portion of the one connection linear portions. The connection portions connect the terminal ends of the axial linear portions and the one end side vertices of the annular bodies adjacent to the annular bodies having the axial linear portions on the other end side in the axial direction of the stent.

In particular, in the plurality of annular bodies located in the central portion of the stent, in the expanded state of the stent, one of the connection linear portions includes axial linear portions having one end side vertices of the one end side bent portions as starting ends, extending so as to be parallel to the axial direction of the stent as compared with the connection linear portions, and having a shorter axial length than the axial length of the annular bodies, and bent portions having starting ends at terminal ends of the axial linear portions, extending in a circumferential direction of the stent and in a direction away from the one end side vertices, and having terminal ends connected to the remaining portion of the one of the connection linear portions. The connection portions connect the terminal ends of the axial linear portions and the one end side vertices of the annular body adjacent to the annular body having the axial linear portions on the other end side in the axial direction of the stent.

Thus, one ends of the connection portions are connected to the terminal ends of the axial linear portions of the annular bodies and are not connected to vertices of the bent portions of the annular bodies. Thus, the linear components do not intersect in a cross shape at the connection portions as intersections, and the intersections are divided into two branched intersections, which are shifted in the axial direction. Thus, favorable radial deformability is provided at the time of diameter reduction, so that the diameter can be reduced.

The in-vivo indwelling stent of the present disclosure is as follows.

(1) An in-vivo indwelling stent in which a plurality of annular bodies each formed in a circular shape with one endless linear component are arranged in an axial direction, and adjacent annular bodies are connected by connection portions, each of the annular bodies including a plurality of one end side bent portions located on one end side in the axial direction of the stent, a plurality of other end side bent portions located on the other end side in the axial direction of the stent, and a plurality of connection linear portions that connect the one end side bent portions and the other end side bent portions, further, in an expanded state of the stent, one of connection linear portions of the plurality of annular bodies located in a central portion of the stent including axial linear portions having one end side vertices of the one end side bent portions as starting ends, extending so as to be parallel to the axial direction of the stent as compared with the other portion of the connection linear portions, and having a shorter axial length than an axial length of the annular bodies; and bent portions having starting ends at terminal ends of the axial linear portions, extending in a circumferential direction of the stent and in a direction away from the one end side vertices, and having terminal ends connected to a remaining portion of the one of the connection linear portions, and the connection portions connecting the terminal ends of the axial linear portions and the one end side vertices of the annular body adjacent to the annular body having the axial linear portions on the other end side in the axial direction of the stent.

This in-vivo indwelling stent is an in-vivo indwelling stent in which a plurality of annular bodies each formed in a circular shape with one endless linear component are arranged in the axial direction, and adjacent annular bodies are connected by connection portions. The annular body includes a plurality of one end side bent portions located on one end side in the axial direction of the stent, a plurality of other end side bent portions located on the other end side in the axial direction of the stent, and a plurality of connection linear portions that connect the one end side bent portions and the other end side bent portions. Furthermore, in the expanded state of the stent, one of the connection linear portions of the plurality of annular bodies located in the central portion of the stent includes: axial linear portions having one end side vertices of the one end side bent portions as starting ends, extending so as to be parallel to the axial direction of the stent as compared with the other portion of the connection linear portions, and having a shorter axial length than an axial length of the annular bodies; and bent portions having starting ends at terminal ends of the axial linear portions, extending in a circumferential direction of the stent and in a direction away from the one end side vertices, and having terminal ends connected to the remaining portion of the one connection linear portions. The connection portions connect the terminal ends of the axial linear portions and the one end side vertices of the annular bodies adjacent to the annular bodies having the axial linear portions on the other end side in the axial direction of the stent.

In particular, in the plurality of annular bodies located in the central portion of the stent, in the expanded state of the stent, one of the connection linear portions includes axial linear portions having one end side vertices of the one end side bent portions as starting ends, extending so as to be parallel to the axial direction of the stent as compared with the connection linear portions, and having a shorter axial length than the axial length of the annular bodies, and bent portions having starting ends at terminal ends of the axial linear portions, extending in a circumferential direction of the stent and in a direction away from the one end side vertices, and having terminal ends connected to the remaining portion of the one of the connection linear portions. The connection portions connect the terminal ends of the axial linear portions and the one end side vertices of the annular body adjacent to the annular body having the axial linear portions on the other end side in the axial direction of the stent.

Thus, one ends of the connection portions are connected to the terminal ends of the axial linear portions of the annular bodies and are not connected to vertices of the bent portions of the annular bodies. Thus, the linear components do not intersect in a cross shape at the connection portions as intersections, and the intersections are divided into two branched intersections, which are shifted in the axial direction. Thus, favorable radial deformability is provided at the time of diameter reduction, so that the diameter can be reduced.

Furthermore, the above embodiment may be as follows.

(2) The in-vivo indwelling stent according to (1), in which the connection portions include: connection portion parallel portions having one ends at the terminal ends of the axial linear portions and extending substantially parallel to the axial direction of the stent; and connection portion inclined portions extending obliquely in the circumferential direction and the axial direction of the stent from the other ends of the connection portion parallel portions, and terminal ends of the connection portion inclined portions are connected to one end side vertices other than the closest one end side vertices of the adjacent annular body.

(3) The in-vivo indwelling stent according to (1) or (2), in which the axial linear portions are straight-line portions elongated along the axial direction of the stent.

(4) The in-vivo indwelling stent according to (2) or (3), in which the axial linear portions and the connection portion parallel portions of the connection portions have a continuous linear shape.

(5) The in-vivo indwelling stent according to any one of (2) to (4), in which the terminal ends of the connection portion parallel portions of the connection portions are located on the same side as the other end side vertices of the annular body including the axial linear portions to which one ends of the connection portions are connected or slightly closer to the other end side of the stent than the other end side vertices in the axial direction of the stent in an expanded state of the stent, and are located on the same side as the other end side vertices of the annular body including the axial linear portions to which the one ends of the connection portions are connected or slightly closer to the one end side of the stent than the other end side vertices in the axial direction of the stent in a contracted state of the stent.

(6) The in-vivo indwelling stent according to any one of (1) to (5), in which the connection linear portions that connect the terminal ends of the axial linear portions and the other end side vertices of the other end side bent portions are shorter than the connection linear portions that connect the one end side vertices of the one end side bent portions and the other end side vertices of the other end side bent portions.

(7) The in-vivo indwelling stent according to any one of (1) to (6), in which a distance between the one end side vertices and the terminal ends of the axial linear portions or starting ends of the bent portions is 1/16 to ½ of a length of the connection linear portions extending from the one end side vertices.

(8) The in-vivo indwelling stent according to any one of (1) to (7), in which each of the annular bodies located in the central portion of the stent includes a plurality of the bent portions, and the bent portions extend in the same circumferential direction.

(9) The in-vivo indwelling stent according to (8), in which in adjacent annular bodies located in the central portion of the stent, the bent portions adjacent in the axial direction of the stent extend in different circumferential directions.

(10) The in-vivo indwelling stent according to any one of (1) to (9), in which the stent does not have a one end side bent portion that is a free end except for the annular body located at one end.

(11) The in-vivo indwelling stent according to any one of (1) to (10), in which terminal ends of all the axial linear portions of the annular bodies having the axial linear portions are connected to the connection portions.

(12) The in-vivo indwelling stent according to any one of (1) to (11), in which the stent includes: one end portion annular body connection portions that connect the other end side vertices of the annular body located at one end and one end side vertices of the annular body adjacent to the annular body located at the one end; and other end portion annular body connection portions that connect the one end side vertices of the annular body located at the other end and the other end side vertices of the annular body adjacent to the annular body located at the other end, and the connection portions, the one end portion annular body connection portions, or the other end side annular body connection portions are connected to the one end side vertices of all the annular bodies except the annular body located at one end.

(13) The in-vivo indwelling stent according to any one of (1) to (12), in which the stent includes convex portions that are located between adjacent annular bodies and protrude to the outside of the stent in an expanded state of the stent.

(14) The in-vivo indwelling stent according to any one of (1) to (13), in which the stent is formed in a substantially cylindrical shape, is compressed in a central axis direction at the time of in-vivo insertion, and expands outward to restore a shape before compression at the time of in-vivo indwelling.

Further, the stent delivery system of the present disclosure is as follows.

(15) A stent delivery system including: a sheath; the stent according to (14) stored in a distal end portion of the sheath; and a shaft that is to be inserted into the sheath and releases the stent from a distal end of the sheath.

Furthermore, the above embodiment may be as follows.

(16) The stent delivery system according to (15), in which the stent is released from the distal end of the sheath by movement of the sheath to a proximal end side, and the stent partially exposed from the sheath can be restored in the sheath by moving the sheath to a distal end side with respect to the stent.

The detailed description above describes embodiments of an in-vivo indwelling stent and a stent delivery system to be used for improving stenosis or occlusion generated in a body lumen such as a blood vessel, a bile duct, a trachea, an esophagus, or a urethra. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An in-vivo indwelling stent comprising:
a plurality of annular bodies, each of the plurality of annular bodies formed in a circular shape with one endless linear component arranged in an axial direction, and adjacent annular bodies of the plurality of annular bodies being connected by connection portions;
each of the plurality of annular bodies includes a plurality of one end side bent portions located on one end side in the axial direction of the stent, a plurality of other end side bent portions located on the other end side in the axial direction of the stent, and a plurality of connection linear portions that connect the one end side bent portions and the other end side bent portions;
in an expanded state of the stent, one of the plurality of connection linear portions of the plurality of annular bodies located in a central portion of the stent includes:
axial linear portions having one end side vertices of the one end side bent portions as starting ends, extending so as to be parallel to the axial direction of the stent as compared with the other portion of the connection linear portions, and having a shorter axial length than an axial length of the plurality of annular bodies; and
bent portions having starting ends at terminal ends of the axial linear portions, extending in a circumferential direction of the stent and in a direction away from the one end side vertices, and having terminal ends connected to a remaining portion of the one of the connection linear portions; and
the connection portions connecting the terminal ends of the axial linear portions and the one end side vertices of the annular body adjacent to the annular body having the axial linear portions on the other end side in the axial direction of the stent.

2. The in-vivo indwelling stent according to claim 1, wherein the connection portions include:
connection portion parallel portions having one ends at the terminal ends of the axial linear portions and extending substantially parallel to the axial direction of the stent; and
connection portion inclined portions extending obliquely in the circumferential direction and the axial direction of the stent from the other ends of the connection portion parallel portions, and terminal ends of the connection portion inclined portions are connected to one end side vertices other than the closest one end side vertices of the adjacent annular body.

3. The in-vivo indwelling stent according to claim 1, wherein the axial linear portions are straight-line portions elongated along the axial direction of the stent.

4. The in-vivo indwelling stent according to claim 2, wherein the axial linear portions and the connection portion parallel portions of the connection portions have a continuous linear shape.

5. The in-vivo indwelling stent according to claim 2, wherein terminal ends of the connection portion parallel portions of the connection portions are located on the same side as the other end side vertices of the annular body including the axial linear portions to which one ends of the connection portions are connected or slightly closer to the other end side of the stent than the other end side vertices in the axial direction of the stent in an expanded state of the stent, and are located on the same side as the other end side vertices of the annular body including the axial linear portions to which the one ends of the connection portions are connected or slightly closer to the one end side of the stent than the other end side vertices in the axial direction of the stent in a contracted state of the stent.

6. The in-vivo indwelling stent according to claim 1, wherein the connection linear portions that connect the terminal ends of the axial linear portions and the other end side vertices of the other end side bent portions are shorter than the connection linear portions that connect the one end side vertices of the one end side bent portions and the other end side vertices of the other end side bent portions.

7. The in-vivo indwelling stent according to claim 1, wherein a distance between the one end side vertices and the terminal ends of the axial linear portions or starting ends of the bent portions is 1/16 of a length of the connection linear portions extending from the one end side vertices to ½ of the length of the connection linear portions extending from the one end side vertices.

8. The in-vivo indwelling stent according to claim 1, wherein each of the plurality of annular bodies located in the central portion of the stent includes a plurality of the bent portions, and the bent portions extend in the same circumferential direction.

9. The in-vivo indwelling stent according to claim 8, wherein in adjacent annular bodies located in the central portion of the stent, the bent portions adjacent in the axial direction of the stent extend in different circumferential directions.

10. The in-vivo indwelling stent according to claim 1, wherein the stent does not have a one end side bent portion that is a free end except for the annular body located at one end.

11. The in-vivo indwelling stent according to claim 1, wherein terminal ends of all the axial linear portions of the plurality of annular bodies having the axial linear portions are connected to the connection portions.

12. The in-vivo indwelling stent according to claim 1, wherein the stent includes:
one end portion annular body connection portions that connect the other end side vertices of the annular body located at one end and the one end side vertices of the annular body adjacent to the annular body located at the one end;
other end portion annular body connection portions that connect the one end side vertices of the annular body located at the other end and the other end side vertices of the annular body adjacent to the annular body located at the other end, and the connection portions; and
the connection portions, the one end portion annular body connection portions, or the other end portion annular body connection portions are connected to the one end side vertices of all the plurality of annular bodies except an annular body located at one end.

13. The in-vivo indwelling stent according to claim 1, wherein the stent includes convex portions that are located between adjacent annular bodies and protrude to the outside of the stent in an expanded state of the stent.

14. The in-vivo indwelling stent according to claim 1, wherein the stent is formed in a substantially cylindrical shape, is configured to be compressed in a central axis direction at the time of in-vivo insertion, and configured to expand outward to restore a shape before compression at the time of in-vivo indwelling.

15. A stent delivery system comprising:
a sheath;
the stent according to claim 14, the stent configured to be stored in a distal end portion of the sheath; and
a shaft configured to be inserted into the sheath and to release the stent from a distal end of the sheath.

16. The stent delivery system according to claim 15, wherein the stent is released from the distal end of the sheath by movement of the sheath to a proximal end side, and the stent partially exposed from the sheath can be restored in the sheath by moving the sheath to a distal end side with respect to the stent.

17. An in-vivo indwelling stent comprising:
a plurality of annular bodies formed in a circular shape arranged in an axial direction, the plurality of annular bodies including adjacent annular bodies being connected by connection portions;
the plurality of annular bodies including a plurality of one end side bent portions located on one end side in the axial direction of the stent, a plurality of other end side bent portions located on the other end side in the axial direction of the stent, and a plurality of connection linear portions that connect the one end side bent portions and the other end side bent portions;
one of the plurality of connection linear portions of the plurality of annular bodies located in a central portion of the stent includes axial linear portions having one end side vertices of the one end side bent portions as starting ends, extending so as to be parallel to the axial direction of the stent as compared with the other portion of the connection linear portions, and having a shorter axial length than an axial length of the plurality of annular bodies, and bent portions having starting ends at terminal ends of the axial linear portions, extending in a circumferential direction of the stent and in a direction away from the one end side vertices, and having terminal ends connected to a remaining portion of the one of the connection linear portions;
the connection portions connecting the terminal ends of the axial linear portions and the one end side vertices of the annular body adjacent to the annular body having the axial linear portions on the other end side in the axial direction of the stent; and
wherein the connection portions include:
connection portion parallel portions having one ends at the terminal ends of the axial linear portions; and
connection portion inclined portions extending obliquely in the circumferential direction and the axial direction of the stent from the other ends of the connection portion parallel portions, and terminal ends of the connection portion inclined portions are connected to one end side vertices other than the closest one end side vertices of the adjacent annular body.

18. The in-vivo indwelling stent according to claim 17, wherein
the plurality of annular bodies located in the central portion of the stent include a plurality of the bent portions, and the bent portions extend in the same circumferential direction; and
in adjacent annular bodies located in the central portion of the stent, the bent portions adjacent in the axial direction of the stent extend in different circumferential directions.

19. The in-vivo indwelling stent according to claim 17, wherein the axial linear portions and the connection portion parallel portions of the connection portions have a continuous linear shape.

20. An in-vivo indwelling stent comprising:
a plurality of annular bodies including adjacent annular bodies being connected by connection portions;
the plurality of annular bodies including a plurality of one end side bent portions located on one end side in the axial direction of the stent, a plurality of other end side bent portions located on the other end side in the axial direction of the stent, and a plurality of connection linear portions that connect the one end side bent portions and the other end side bent portions;
in an expanded state of the stent, one of the plurality of connection linear portions of the plurality of annular bodies located in a central portion of the stent includes axial linear portions having one end side vertices of the one end side bent portions as starting ends, extending so as to be parallel to the axial direction of the stent as compared with the other portion of the connection linear portions, and having a shorter axial length than an axial length of the plurality of annular bodies, and bent portions having starting ends at terminal ends of the axial linear portions, extending in a circumferential direction of the stent and in a direction away from the one end side vertices, and having terminal ends connected to a remaining portion of the one of the connection linear portions;
the connection portions connecting the terminal ends of the axial linear portions and the one end side vertices of the annular body adjacent to the annular body having the axial linear portions on the other end side in the axial direction of the stent, the connection portions including:
connection portion parallel portions having one ends at the terminal ends of the axial linear portions and extending substantially parallel to the axial direction of the stent; and
connection portion inclined portions extending obliquely in the circumferential direction and the axial direction of the stent from the other ends of the connection portion parallel portions, and terminal ends of the connection portion inclined portions are connected to one end side vertices other than the closest one end side vertices of the adjacent annular body; and
wherein the axial linear portions and the connection portion parallel portions of the connection portions have a continuous linear shape.
